(19) United States
(12) Patent Application Publication
(10) Pub. No.: US 2018/0329045 A1
(43) Pub. Date: Nov. 15, 2018

(54) FLEXIBLE ULTRASOUND SYSTEM FOR BOTH IMAGING AND HIGH POWER DELIVERY APPLICATIONS

(71) Applicant: GENERAL ELECTRIC COMPANY, Schenectady, NY (US)

(72) Inventors: Han Peng, Niskayuna, NY (US); John Glaser, Niskayuna, NY (US); Kieran Wall, Niskayuna, NY (US); Juan Sabate, Niskayuna, NY (US)

(73) Assignee: GENERAL ELECTRIC COMPANY, Schenectady, NY (US)

(21) Appl. No.: 15/593,370

(22) Filed: May 12, 2017

(51) Int. Cl. G01S 7/52; A61B 8/00

(52) U.S. Cl. CPC G01S 7/5208; G01S 7/5202; A61B 8/4477

(57) ABSTRACT

There is set forth herein an ultrasound signal generating circuit having a control circuit which can include a control circuit having a control signal generating circuit; a low power signal driver circuit for providing a low power ultrasound signal; a high power signal driver circuit for providing a high power ultrasound signal; and a radiofrequency switch configured to transmit the low power ultrasound signal and the high power ultrasound signal to the ultrasound probe, wherein the radiofrequency switch isolates the low power signal driver circuit from the high power signal driver circuit; wherein the ultrasound signal generating circuit is configured so that the radiofrequency switch transmits an output low power ultrasound signal output by the low power signal driver circuit or an output high power ultrasound signal output by the high power signal driver circuit based on an output of the control signal generating circuit.

### GOVERNMENT RIGHTS STATEMENT

This invention was made with government support under NASA of the United States of America, under government contract number NNC09BA02B and subcontract number SPACEDOC 2013-003. The government may have certain rights in the invention.

### BACKGROUND

The subject matter disclosed herein relates to ultrasound systems that may be utilized for diagnostic and/or therapeutic purposes. More particularly, present embodiments are directed to systems and methods that facilitate adjustment of a focus depth density and acoustic energy of an ultrasound probe between multiple positions while maintaining acoustic coupling.

The term ultrasound generally refers to cyclic sound pressure that has a frequency in a range that is higher than the upper limit of human hearing. A typical ultrasound frequency may include 1 to 20 megahertz. Ultrasound is frequently used for imaging purposes. For example, ultrasound is used in ultrasonography, which is a medical imaging technique that emits high frequency sound waves into a patient's body and detects echoes of the sound waves to produce images of features internal to the patient's body (e.g., blood flow images and intrauterine images). However, ultrasound may also be utilized to perform functions. For example, ultrasound for therapeutic purposes (e.g., stimulating a damaged muscle).

While there are numerous uses for ultrasound, a representative example may include a medical imaging application. In a typical ultrasound imaging application, sound waves are emitted into a patient's body from a probe and are reflected back to the probe when they hit boundaries. For example, some waves may reflect back to the probe upon reaching a boundary between fluid and tissue and other waves may reflect back to the probe upon reaching a boundary between tissue and bone. The probe detects the reflected waves and relays them to a monitor that utilizes the speed of the ultrasound and the time required to detect the reflected wave relative to the time of emission to calculate the distance from the probe to the reflecting surface. The distances and intensities of the detected waves may then be displayed to provide an image of the observed tissue. Relatively dense tissue may be distinguished from less dense tissue based on a difference in intensity because more dense tissue may reflect more ultrasound waves than less dense tissue.

### BRIEF DESCRIPTION

There is set forth herein an ultrasound signal generating circuit having a control circuit which can include a control circuit having a control signal generating circuit; a low power signal driver circuit for providing a low power ultrasound signal; a high power signal driver circuit for providing a high power ultrasound signal; and a radiofrequency switch configured to transmit the low power ultrasound signal and the high power ultrasound signal to the ultrasound probe, wherein the radiofrequency switch isolates the low power signal driver circuit from the high power signal driver circuit; wherein the ultrasound signal generating circuit is configured so that the radiofrequency switch transmits an output low power ultrasound signal output by the low power signal driver circuit or an output high power ultrasound signal output by the high power signal driver circuit based on an output of the control signal generating circuit.

### DRAWINGS

These and other features, aspects, and advantages of the present invention will become better understood when the following detailed description is read with reference to the accompanying drawings in which like characters represent like parts throughout the drawings, wherein:

FIG. 1 is a block diagram of one element of ultrasound pulse generating system shown in the environment of an ultrasound system in which the ultrasound signal generating circuit to drive one or more ultrasound probes;

FIG. 2 is a flowchart illustrating a synchronization method that can be performed by an ultrasound signal generating circuit in one embodiment;

FIG. 3 is a physical from view of an ultrasound system according to one embodiment;

FIG. 4 illustrates a displayed user interface that can be displayed on a display of an ultrasound system according to one embodiment;

FIG. 5 is a schematic circuit diagram illustrating the output selection/transmission components of an ultrasound pulse generating circuit according to one embodiment;

FIG. 6 is a timing diagram illustrating various timing and control features of an ultrasound system.

### DETAILED DESCRIPTION

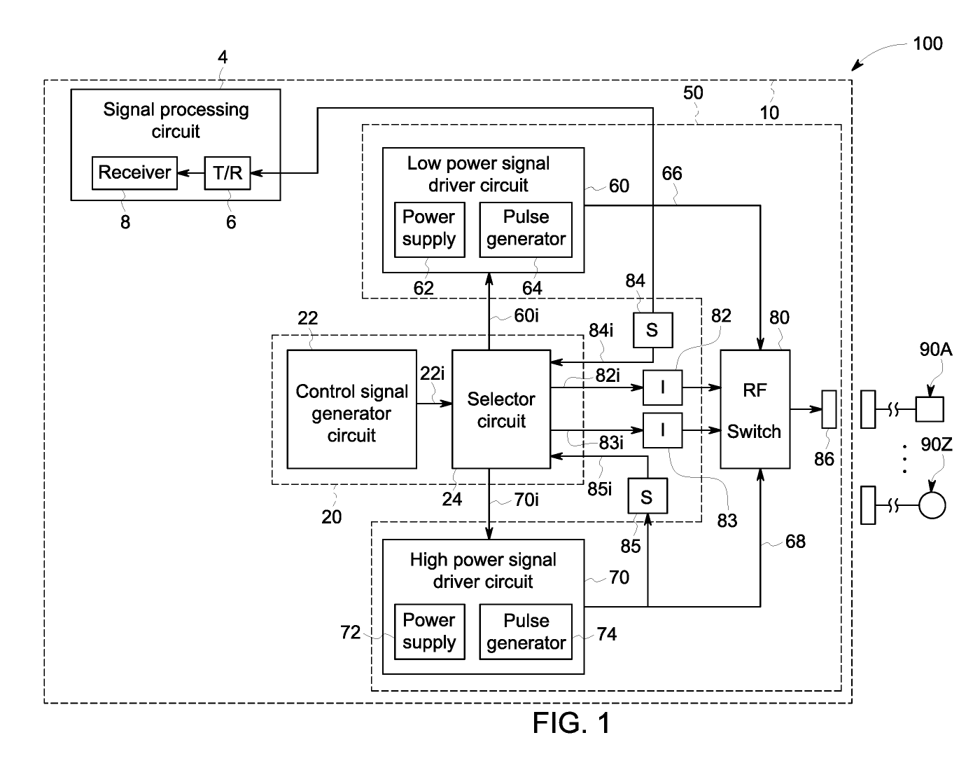

There is illustrated in FIG. 1, a circuit block diagram illustrating an ultrasound signal generating circuit 10. Ultrasound signal generating circuit 10 can be incorporated in an ultrasound system 100, wherein ultrasound signal generating circuit 10 drives one or more candidate ultrasound probes, such as one or more ultrasound probes 90A-90Z. In one embodiment, there can be attached a single ultrasound probe 90A e.g. a probe of candidate probes 90A-90Z at a given time. As set forth herein, ultrasound signal generating circuit 10 can include features that facilitate operation of ultrasound system 100 in multiple operating modes including e.g. imaging operating modes of operation and therapeutic high power modes of operation. In general, when operating to support imaging low power modes of operation, ultrasound signal generating circuit 10 can output relatively lower power probe drive signals. When operating to support therapeutic high power modes of operation ultrasound signal generating circuit 10 can output relatively higher power probe drive signals. Ultrasound signal generating circuit 10 can include an associated signal processing circuit 4 for processing return signals. Signal processing circuit 4 can be active in an imaging low power modes of operation to process return signals attributed to reflected ultrasound signals reflected from target e.g. body tissue being subject to imaging. Return signals can be picked up by a transducer array of an attached ultrasound probe.

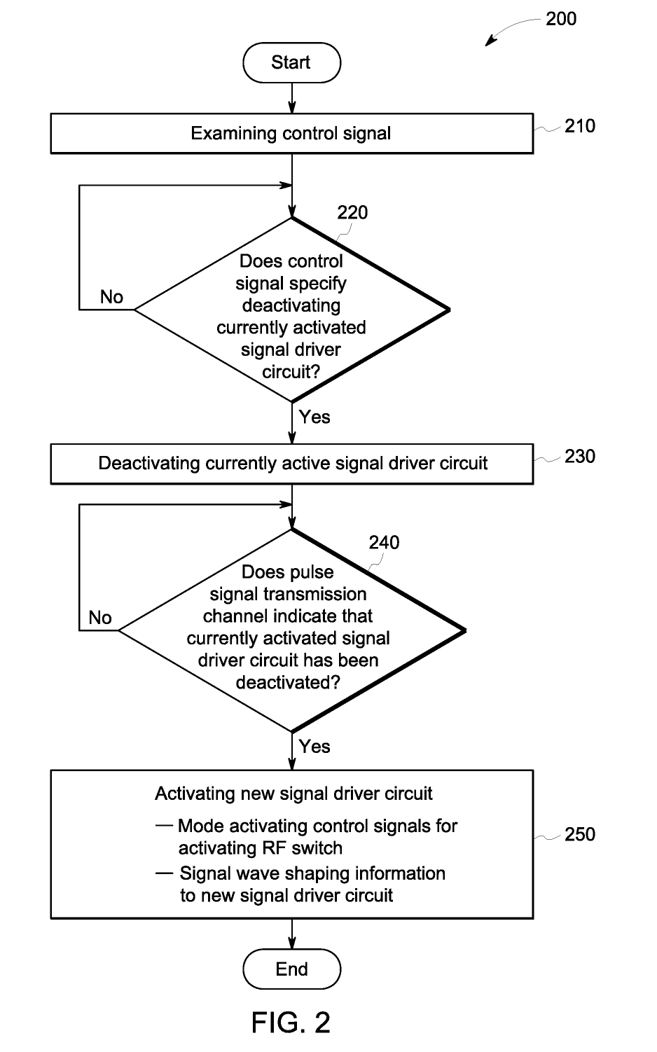

In one embodiment as set forth in FIG. 1, ultrasound signal generating circuit 10 can include control circuit 20 and driver circuit 50. Control circuit 20 in one embodiment can include control signal generator circuit 22 and selector circuit 24, which selector circuit 24 in one embodiment can be provided by field programmable gate array (FPGA). Driver circuit 50 can include a low power signal driver circuit 60, and high power signal driver circuit 70, as well as radiofrequency (RF) switch 80, and interface 86. Low power signal driver circuit 60 can include a power supply 62 and pulse generator 64. High power signal driver circuit 70 can include a power supply 72, logically and physically separate from power supply 62 and pulse generator 64. In a further aspect, ultrasound signal generating circuit 10 can include isolator circuits 82 and 83 for isolating driver circuit 50 from control circuit 20, and can also include synchronization circuits 84 and 85 for synchronization of mode transitions performed by ultrasound signal generating circuit 10. Operational aspects of synchronization circuits 84 and 85 are described further in reference to the flowchart of FIG. 2 illustrating a synchronization method that can be performed by ultrasound signal generating circuit 10. “Low power” and “high power” are used as relative terms. Elements set forth herein prefaced with the terms “low” and “high” can alternatively be prefaced with the terms “first” and “second”. For example, “low power signal driver circuit” can alternatively be termed a “first power signal driver circuit” and “high power signal driver circuit” can alternatively be termed a “second power signal driver circuit.” In one embodiment, low power signal driver circuit 60 outputs relatively lower power probe drive signal than high power signal circuit 70. In one embodiment, low power signal driver circuit 60 outputs relatively lower average power probe drive signal than high power signal circuit 70. The lower power probe drive signal can be provided by a low power pulse signal as set forth herein. The higher power probe drive signal can be provided by a high power pulse signal as set forth herein. There is set forth herein a flexible ultrasound image system for both imaging and high power delivery applications.

At block 210 of method 200, ultrasound signal generating circuit 10 e.g. by selector circuit 24, can examine a current control signal generated by control signal generator circuit 22. Control signal generator circuit 22 can be configured to generate control signals e.g. based on user defined data defined using a manually operating user interface and/or based on a probe currently attached to interface 86.

For performance at block 210 in one embodiment, selector circuit 24 can examine a current control signal as received via control signal input signal line 22i. Based on the examining performed at block 210, selector circuit 24 at block 220 to determine whether the currently active control signal specifies deactivating the currently activated signal driver circuit. In the operation of ultrasound signal generating circuit 10 one of low power signal driver circuit 60 and high power signal driver circuit 70 can be active.

Ultrasound signal generating circuit 10 can be operative to perform mode changes to transition modes of operation of ultrasound signal generating circuit 10. In one aspect ultrasound signal generating circuit 10 can transition from a low power state in which low power signal driver circuit 60 is active and high power signal driver circuit 70 is inactive to a high power state in which high power signal driver circuit 70 is active and low power signal driver circuit 60 is inactive. In one aspect ultrasound signal generating circuit 10 can perform a state transition from a high power state in which high power signal driver circuit 70 is active and low power signal driver circuit 60 is in active to a low power state in which low power signal driver circuit 60 is active and high power signal driver circuit is inactive. Referring to blocks 210 and 220, selector circuit 24 can execute blocks 210 and 220 iteratively in a loop, until the current control signal specifies a mode change.

On the determination that a currently active control signal specifies mode transition, selector circuit 24 can proceed to block 230. Based on a current control signal specifying deactivating a currently activated signal driver circuit, selector circuit 24 can proceed to block 230 to perform deactivating the current active signal driver circuit e.g. can deactivate low power signal driver circuit 60 if low power signal driver circuit 60 is currently active or can deactivate high power signal driver circuit 70 if high power signal driver circuit 70 is currently active. Selector circuit 24 can communicate a deactivation signal via a signal line 60i for deactivating low power signal driver circuit 60.

For deactivating high power signal driver circuit 70, selector circuit 24 can transmit a deactivation signal via signal line 70i. According to synchronization method 200, selector circuit 24 can be operative to transmit signal driver circuit deactivating signals prior to transmitting signal driver circuit activation signals and can be restricted from transmitting an activation signal to activate a signal driver circuit until a time that selector circuit 24 confirms that a currently active signal driver circuit has been successfully deactivated.

At block 240, selector circuit 24 can examine a driver signal transmission channel e.g. an appropriate one of transmission channel 66 or transmission channel 68 to ascertain whether the channel indicates that the drive signal has been removed from the channel. For performance of block 240, selector circuit 24 can monitor an output received from an appropriate one of synchronization circuit 84 or 85. An output from synchronization circuit 84 can be received via signal line 84i and an output of synchronization circuit 85 can be received via signal line 85i. An output of synchronization circuit 84 can be monitored by selector circuit 24 in the case that low power signal driver circuit 60 is currently active and is being deactivated. An output from synchronization circuit 85 can be monitored in the case that high power signal driver circuit 70 is currently active and is being deactivated. Selector circuit 24 can be configured so that selector circuit 24 is restricted from transmitting an activation signal to activate a new signal driver circuit 60 or 70 until such time that selector circuit 24, via monitoring of an output of synchronization circuit 84 or 85 confirms that a currently activated low power signal driver circuit 60 or high power signal driver circuit 70 has been successfully deactivated.

Referring to synchronization circuit 84 and synchronization circuit 85, synchronization circuits 84 and 85 can include appropriate circuitry to sense whether low power pulse signal transmission channel 66 or high power pulse signal transmission channel 68 is currently transmitting a pulse signal for driving an ultrasonic probe. Synchronization circuits 84 and 85 can include appropriate current limiting circuitry so that current conducted through synchronization circuits 84 and 85 is reduced to safe low levels so as to avoid a negative effect on selector circuit 24. Control circuit 20 including control signal generator circuit 22 and selector circuit 24 in one embodiment can be provided by digital circuitry that can be powered at low voltage levels e.g. 5 volt control voltage supply or less. In one embodiment, control signal generator circuit 22 can be provided by a processor based circuit. Selector circuit 24 can provided by an FPGA in communication with control signal generator circuit 22.

Based on the appropriate one of transmission channel 66 or transmission channel 68 no longer transmitting a drive signal, selector circuit 24 can proceed to block 250 to perform activating a new signal driver circuit e.g. low power signal driver circuit 60 or high power signal driver circuit 70. For performing block 250, selector circuit 24 can transmit a trigger signal for changing the state of RF switch 80 e.g. from high power active to low power active or from low power active to high power active. At block 250, selector circuit 24 can transmit one or more trigger signal for triggering switching of RF switch 80. Selector circuit 24 can transmit a trigger signal provided by a low power mode activating control signal for activating low power signal driver circuit 60 via signal line 82i which trigger signal can be received by isolator circuit 82, which can pass the trigger signal provided by a low power mode activating control signal for receipt by RF switch 80. Selector circuit 24 can transmit a trigger signal provided by a high power mode activating control signal for activation of high power signal driver circuit 70 via signal line 83i for receipt by isolator circuit 83, which isolator circuit 83 can pass the trigger signal provided by a high power mode activating control signal to RF switch 80. At block 250, selector circuit 24 via an appropriate one of signal lines 60i or 70i with an activation signal transmitted by selector circuit 24 to an appropriate one of low power signal driver circuit 60 or high power signal driver circuit 70 at block 250. There can be further transmitted waveform information via signal line 60i or 70i that specifies e.g. pulse with frequency and/or amplitude information to the appropriate signal driver circuit 60 or 70.

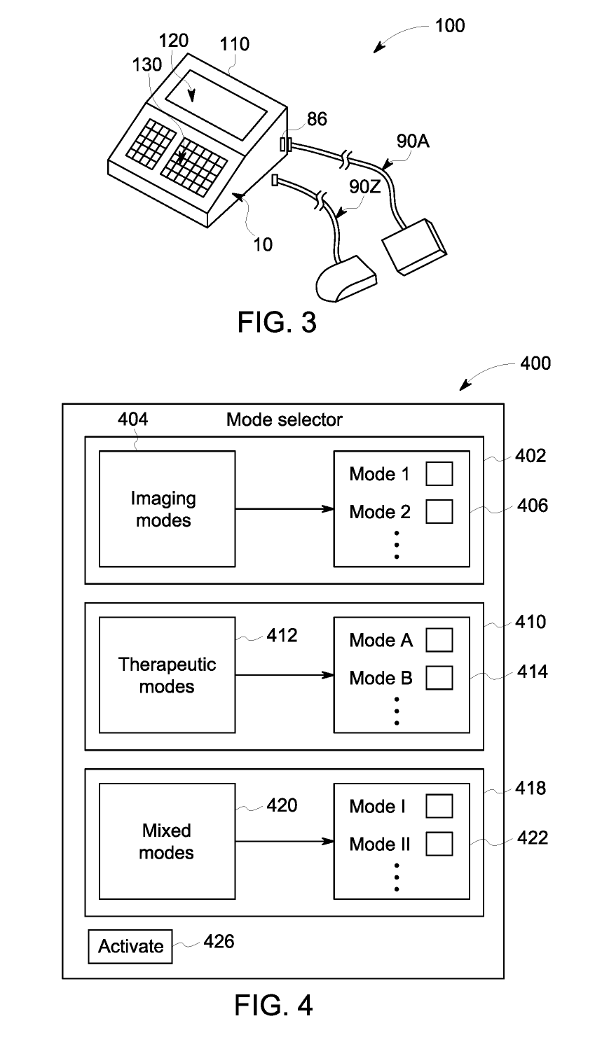

A physical form view of an ultrasound system 100 in one embodiment is illustrated in reference to FIG. 3. Ultrasound system 100 can include one or more housing such as housing 110 that houses ultrasound signal generating circuit 10. Housing 110 in one embodiment can support interface 86 (FIG. 1) to which an ultrasound probe of the set of candidate ultrasounds probes 90A-90Z can be attached. Ultrasound system 100 can also include a display 120 supported by one or more housing e.g. housing 110 and a keyboard 130 supported by one or more housing e.g. housing 110.

A user interface 400 that can be displayed on display 120 is illustrated with reference to FIG. 4. In one embodiment a user can use a user interface 400 to select modes of operation of ultrasound system 100. A user of user interface 400 can be e.g. a healthcare provider user e.g. physician, physician assistant, nurse, or an individual using ultrasound system 100 for personal use.

Referring to user interface 400, a user can use area 402 to select imaging low power modes of operation. A user can activate button 404 to initiate display of imaging low power mode menu options in area 406 and can select between imaging low power modes e.g. mode 1, mode 2, etc. using area 406. A user can activate button 412 to initiate display of therapeutic high power mode selector area 414 and can select between different therapeutic high power modes e.g. therapeutic high power mode A, Mode B, etc. using area 414. Using area 408 a user can select mixed modes of operation e.g. modes in which ultrasound system 100 can automatically transition between imaging low power mode operation and therapeutic high power mode operation. When a mixed mode is active ultrasound signal generating circuit 10 can automatically transition between states of operation e.g. between a low power state in which low power signal driver circuit 60 is active and high power signal driver circuit 70 is in active and a high power state in which high power signal driver circuit 70 is activate and low power signal driver circuit 60 is inactive. A user can activate button 420 to call up area 422 that displays indicators of various mixed operating modes e.g. Mixed Mode i and Mixed Mode ii, and a user can select between various mixed mode operating modes using area 422. A user can activate a selected operating mode using activate button 426. By way of its high rate switching operation RF switch 80 can facilitate a variety of mixed mode operations in which ultrasound system 100 can automatically and iteratively transition between a first state wherein low power signal driver circuit 60 is ON and high power signal driver circuit 70 is OFF and a second state wherein high power signal driver circuit 70 is ON and low power signal driver circuit 60 is OFF.

In one embodiment, a control signal that can be generated by control signal generator circuit 22 can be based on user defined mode selection data which can be defined by a user using a manually operated user interface 400 as set forth in reference to FIG. 4.

In addition or alternatively, the control signal that can be generated by control signal generator circuit 22 can be based on a probe that is currently attached to interface 86 as set forth in FIG. 1. For example, based on a first probe e.g. probe 90A being attached to interface 86, control signal generator circuit 22 can generate a first control signal. Based on a second probe e.g. probe 90Z being attached to interface 86, control signal generator circuit 22 can generate a second control signal. Ultrasound signal generating circuit 10 can be configured so that one ultrasound probe of a candidate set of ultrasound probes 90A-90Z can be attached to interface 86 at a given time.

A set of probes e.g. probes 90A-90Z can include one or more imaging probe and one or more therapeutic probe. Based on an imaging probe being attached to interface 86, control signal generator circuit 22 can generate a control signal for initiating operation in an imaging low power mode. Based on a therapeutic probe being attached to interface 86, control signal generator circuit 22 can generate a control signal for initiating operation in a therapeutic high power mode. A control signal initiating an imaging low power mode can initiate deactivating of high power signal driver circuit 70 is currently active and can initiate activating low power signal driver circuit 60. A control signal initiating a therapeutic high power mode can initiate deactivating of low power signal driver circuit 60 if currently active and can initiate activating high power signal driver circuit 70.

A set of probes 90A-90Z can include one or more imaging probe adapted for use with an imaging low power mode of operation, one or more therapeutic probe adapted for use with a therapeutic high power mode of operation, and one or more mixed mode probe adapted for use with a mixed mode of operation in which ultrasound system 100 automatically transitions between and imaging low power mode and a therapeutic high power mode. In one embodiment, attachment of an imaging probe can restrict modes of operation that can be selected by a user. For example, referring to user interface 400, on the attachment of an imaging probe, the modes depicted in area 410 and 418 of user interface 400 can be restricted and made not available for selection by a user. Similarly, on the attachment of a therapeutic probe, the modes of operation depicted in area 402 and 418 of user interface 400 can be restricted and not available for selection by a user. On the attachment of a mixed mode probe, which can include probe transducers adapted for use in any one of an imaging low power mode, a therapeutic high power mode, and a mixed mode, some modes of operation depicted in each of area 402, 410 and 418 can be restricted and others made available for selection.

An imaging low power mode of operation can be provided by a non-harmonic imaging low power mode of operation or a harmonic imaging low power mode of operation. With non-harmonic imaging, ultrasound system 100 can transmit and receive a sound pulse of a specific frequency. The difference between the transmitted and returned signal is that the returned signal is less intense, losing strength as it passes through tissue. With harmonic imaging the signal returned by the tissue includes not only the transmitted “fundamental” frequency, but also signals of other frequencies. Most notably, the “harmonic” frequency, which is twice the fundamental frequency. Once this combined fundamental/harmonic signal is received, the ultrasound system separates out the two components and then processes the harmonic signal alone. Therapeutic operating modes can be performed with or without processing of a return signal. Therapeutic ultrasound probes of ultrasound probes 90A-90Z can be specially adapted for a variety of alternative applications, e.g. physical therapy, hyperthermia, extracorporeal shock wave lithotripsy, intracorporeal lithotripsy high intensity focused ultrasound, harmonic scalpels, skin permeabilization, bone fracture healing.

Ultrasound probes 90-90Z that are adapted for imaging typically include one or two dimensional arrays of ultrasound transducers. Linear ultrasound imaging transducer arrays can be provide in various array sizes, e.g. 64×1, 128×1, 256×1, 512×1. Two dimensional ultrasound imaging transducer arrays can be provide in various array sized e.g. 32×32, 64×64, 128×128, 256×256, 512×512. Ultrasound probes 90-90Z that are adapted for therapeutic applications typically include a smaller number of larger scale transducers, and typically include from 1 to about 10 transducers. In general, when operating to support imaging low power modes of operation, ultrasound signal generating circuit 10 can output relatively lower power probe drive signals. When operating to support therapeutic high power modes of operation ultrasound signal generating circuit 10 can output relatively higher power probe drive signals. In typical imaging low power modes, low power signal driver circuit 60 can be operative to drive a transducer array of an imaging ultrasound probe to emit energy at an average energy rating of 5 W or less and in many cases 1 W or less. In typical therapeutic high power modes, circuit 70 can be operative to drive a transducer or transducer array of an imaging ultrasound probe to emit energy at an average energy rating of 5 W or more in some cases 50 W or more.

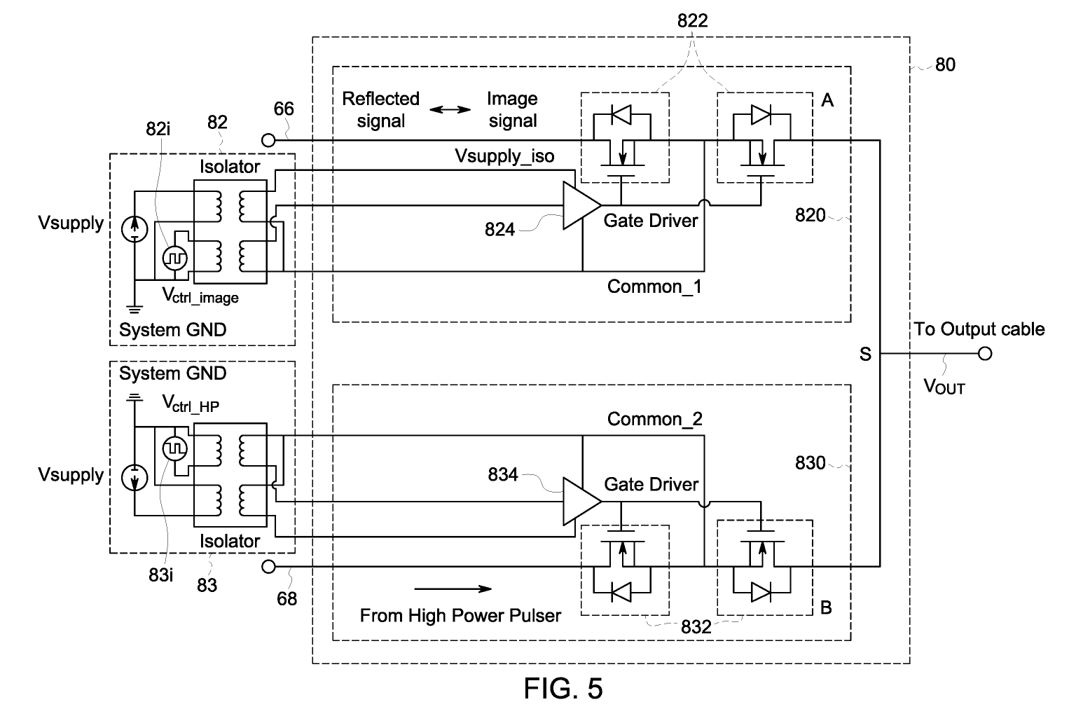

Further details of ultrasound signal generating circuit 10 in one embodiment are described in reference to FIG. 5, illustrating various features in one embodiment of isolator circuit 82, isolator circuit 83, and RF switch 80. RF switch 80 can include a low power transmission area 820 and a high power transmission area 830. Low power transmission area 820 can include a transistor pair 822 provided by a power transistor pair and a gate driver 824. High power transmission area 830 can include transistor pair 832 provided by a power transistor pair and a gate driver 834. In one embodiment each of the first transistor pair 822 and the second transistor pair 832 can be configured to provide bi-directional power flow.

RF switch 80 can be configured so that when a gate driver signal controlling gate driver 824 is on, low power signal area 820 is active so that a pulse signal probe driven by low power signal driver circuit 60 for driving an attached ultrasound probe can be transmitted through RF switch 80. RF switch 80 can be configured so that when a gate driver signal driving gate driver 834 is ON, high power area 830 is active so that RF switch 80 transmits the pulse signal provided by high power signal driver circuit 70 for driving an attached ultrasound probe. A gate driver signal controlling gate driver 824 can be provided by a trigger signal which trigger signal can be provided by a low power mode activating control signal transmitted by selector circuit 24 as previously set forth herein and transmitted via signal line 82i and received by isolator circuit 82 for transmission to gate driver 824. A gate driver signal for driving gate driver 834 can be provided by a trigger signal which trigger signal can be provided by a high power mode activating control signal transmitted by selector circuit 24 via signal line 83i and received by isolator circuit 83 for transmission to gate driver 834. Gate drivers 824 and 834 can be provided by analog gate drivers that are configured to amplify received control signals, which can include a low power mode activating control signal as received by gate driver 824 or a high power mode activating control signal as received by gate driver 834. As set forth herein RF switch 80 can include a first power transistor pair 822 and a second power transistor pair 832, wherein respective analog gate drivers 824 and 834 are used to amplify a control signal provided by respective isolator circuits 82 and 83 to drive the first power transistor pair 822 and the second power transistor pair 832.

RF switch 80 can be configured to provide isolation between components and signals of low power signal driver circuit 60 and high power signal driver circuit 70. Components of RF switch 80 providing isolation can include transistor pair 822 of low power areas 820 and transistor pair 832 of high power area 830. RF switch 80 can provide isolation between low power signal driver circuit 60 and high power signal driver circuit 70, e.g. isolation between propagating pulse signals (high or low power) and circuit elements of the inactive drive circuit side (low or high power).

Transistor pair 822 can be a high frequency low distortion power transistor configured with sources connected together in a back to back configuration and with gates and sources connected together and sources defining a middle (floating) point of the transistor pair. Transistor pair 822 can be configured so that when transistor pair is 822 turned on, a low impedance path is generated to ensure high frequency, low distortion transmission of low power ultrasound signals as well as return signal back from an attached ultrasound probe for processing by signal processing circuit 4. RF switch 80 can be configured to provide isolation for signal processing circuit 4 from a high power pulse signal propagating on transmission channel 68 with good attenuation (e.g. 40 dB attenuation or more). RF switch 80 can be configured to transmit a return signal from an attached ultrasound probe with good linearity and low distortion to provide a low amplitude and low power return signals. Transistor pair 822 can be configured so that when transistor pair 822 is turned off, a high impedance path is generated to provide reliable isolation of output drive signals between high power signal driver circuit 70 and circuit elements of low power signal driver circuit 60 and the signal processing circuit 4. High voltage power transistors that are e.g. Si based or GaN based can be used. A gate driver 824 which can be provided as a high frequency gate driver can be configured to drive transistor pair 822.

Transistor pair 832 can be a high frequency low distortion power transistor pair configured with sources connected together in a back-to-back configuration and with gates and sources connected together and sources defining a middle (floating) point of the transistor pair. Transistor pair 832 can be configured so that when transistor pair 832 is turned on, a low impedance path is generated to ensure high frequency, low distortion transmission of high power ultrasound signals. Transistor pair 832 can be configured so that when transistor pair 832 is turned off, a high impedance path is generated to provide reliable isolation of output drive signals between high power signal driver circuit 70 and from circuit elements of low power signal driver circuit 60 as well as signal processing circuit 4. High voltage power transistors that are e.g. Si based or GaN based can be used. A high frequency gate driver as 834 can be provided to drive transistor pair 822.

Isolator circuit 82 and isolator circuit 83 can provide isolation between RF switch 80 and control circuit 20 so that components of control circuit 20 and control signals thereof are not negatively affected by pulse signals for driving an ultrasound probe propagating along transmission channel 66, transmission channel 68 and/or RF switch 80.

In one embodiment, isolator circuits 82 and 83 provide isolated supply voltage and isolated control signals for RF switch 80. In one embodiment each power transistor pair 822 and 823 can be provided with a transistor source connected together in a back to back configuration. For proper operation with such a configuration, the sources of each power transistor pair 822 and 832 can be connected at a common floating voltage node, e.g. Common_1 and Common_2. Floating voltages at the common floating voltage node (Common_1 or Common_2) can thus include the line voltage of low power pulse signal transmission channel 66 or high power pulse signal transmission channel 68 when the respective transistor pair (822 or 823) is ON.

Isolator circuits 82 and 83 can be provided to input respective control signals to a respective transistor pairs 822 and 823 in a manner that avoids tying a transistor terminal to system ground (which would then tie drive signals to ground). An output of each isolator circuit 82 and 83 can be output to a floating voltage common node, Common_1 and Common_2. The floating voltage common node Common_1 can be connected to sources of first and second transistors defining transistor pair 822. The floating voltage Common_2 can be connected to sources of first and second transistors defining transistor pair 832. Connected sources of each transistor pair 822 and 832 can thus be configured to propagate the line voltage of the transmission channel (transmission channel 66 or transmission channel 68) associated to the transistor pair (822 or 823) when the transistor pair (822 or 832) is ON. Transistor pair 822 can include connected sources and gates and transistor pair 832 can include connected sources and gates. Isolator circuits 82 and 83 can include low voltage isolation transformers in one embodiment.

RF switch 80 in one embodiment can be provided by a single pole double throw switch. In one embodiment, RF switch 80 can be provided by a bidirectional RF switch. RF switch 80 can be provided to be high power handling e.g. in one embodiment can handle power loads of 1 W or more and other embodiments can handle power loads of 5 W or more and in another embodiment can handle power loads of 100 W or more. In one embodiment, RF switch 80 can be provided to have low resistance and can be provided to feature low conduction loss. In one embodiment, RF switch 80 can be a low distortion RF switch. In one embodiment, RF switch 80 can be a high speed RF switch. In one embodiment, RF switch 80 can provide a wide bandwidth.

As set forth in the embodiment described herein in reference to FIG. 5, RF switch 80 can include custom designed analog circuit components. In one embodiment, RF switch 80 can include a diode T/R bridge. In one embodiment, RF switch 80 include a PIN diode switch. In one embodiment, RF switch 80 can include a relay, such as be provided by a G6K-2F-RF relay of the type available from OMRON. In one embodiment, RF switch 80 can include a MEMS relay. In one embodiment, RF switch 80 can include an OTS analog switch.

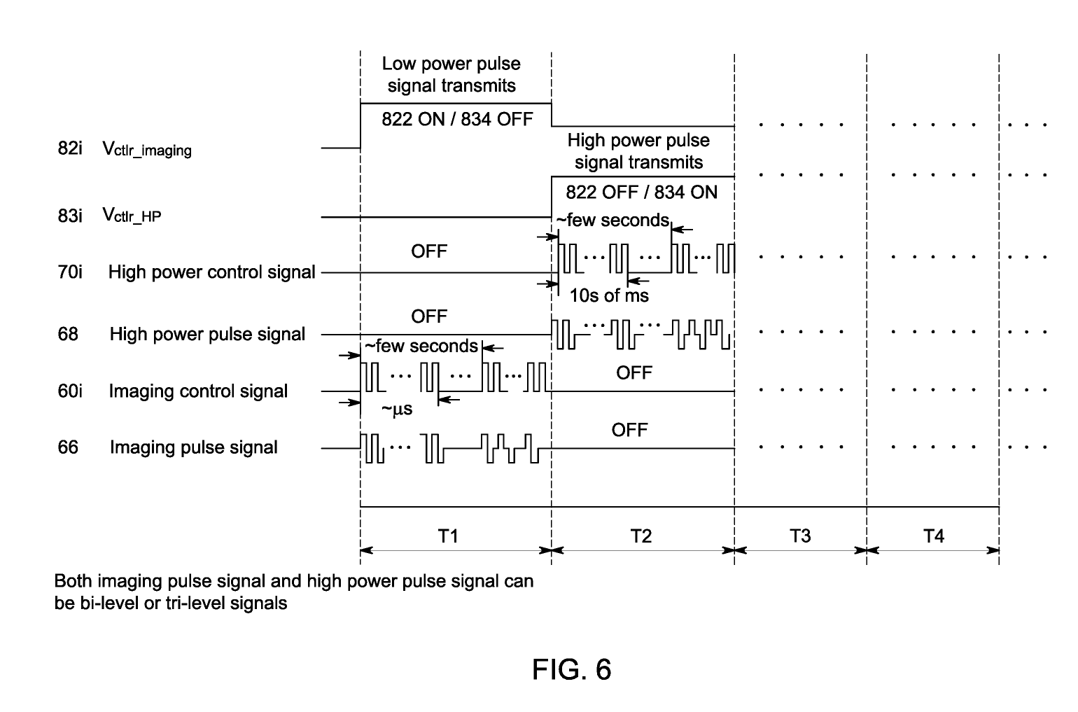

Timing features of ultrasound system 100 are described with reference to the timing diagram of FIG. 6. At time period T1, an imaging low power mode activating control signal on signal line 82i can be ON to drive transistor pair 822 ON so that an imaging low power mode is activated. At time period T1, a high power mode activating control signal on signal line 83i can be OFF to drive transistor pair 832 OFF, so that a high power mode is in a deactivated state. At time period T1 a high power mode activating control signal on signal line 70i can be OFF and with transistor pair 832 OFF, a high power drive signal can be absent from high power drive signal transmission channel. At time period T1 an imaging low power mode activating control signal on signal line 60i can be ON and with transistor pair 822 ON a low power drive signal can be propagating on low power pulse signal transmission channel 66.

At time period T2, an imaging low power mode activating control signal on signal line 82i can be OFF to drive transistor pair 822 OFF so that an imaging low power mode is in a deactivated state. At time period T2, a high power mode activating control signal on signal line 83i can be ON to drive transistor pair 832 ON to activate a high power mode. At time period T2 high power mode activating control signal on signal line 70i can be ON and with transistor pair 832 ON for activating a high power mode a high power pulse signal for driving an ultrasound probe can be propagating on high power pulse signal transmission channel 68. At time period T2 an imaging low power mode activating control signal on signal line 60i can be OFF and with transistor pair 822 OFF a low power pulse signal can be absent from low power pulse signal transmission channel 66.

At time period T3, the states indicated for lines 82i, 83i, 70i, 68, 60i, 66 during time period T1 can be repeated. At time T4, the states indicated for lines 82i, 83i, 70i, 68, 60i, 66 can during time period T3 can be repeated. The states of lines 82i, 83i, 70i, 68, 60i, 66 can transition between the Time T1 states and the time T2 states iteratively and indefinitely. In one embodiment the state transitions between times T1 and T2, and T2 and T3, and T3 and T4 etc. can be in response to a user action e.g. configuration data entered into manually operated user interface 400 (FIG. 4) or a new probe attached to interface 86 (FIG. 4). In one embodiment, the state transitions between time periods T1 and T2, and time periods T2 and T3, and T3 and T4 etc. can be automated, e.g. in a described Mixed Mode (FIG. 4) the states of lines 82i, 83i, 70i, 68, 60i, 66 can iteratively and automatically transition between the time period T1 states and time period T2 states. During periods in which an imaging low power mode is active, e.g. time period T1 and time period T3, T5 etc. signal processing circuit 4 can be receiving and processing a return signal attributable to a reflected ultrasound signal. In some embodiments timing of an output of a low power pulse signal output to transmission channel 66 can be coordinated with active periods of signal processing circuit 4. For example time periods T1, T3, T5 etc. in some embodiments can be characterized by stop periods wherein output of a low power pulse signal by low power signal driver circuit 60 (FIG. 1) for driving an ultrasound probe is stopped temporarily and wherein during the stop periods receipt of a return signal by signal processing circuit 4 is performed. In some embodiments signal processing circuit 4 can be active without low power signal driver circuit 60 being stopped. For example signal processing circuit 4 in some embodiments can filter an output low power pulse signal propagating on low power pulse signal transmission channel 66 that includes return signal components and can process the return signal components.

This written description uses examples to disclose the invention, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

It is to be understood that the above description is intended to be illustrative, and not restrictive. For example, the above-described embodiments (and/or aspects thereof) may be used in combination with each other. In addition, many modifications may be made to adapt a particular situation or material to the teachings of the various embodiments without departing from their scope. While the dimensions and types of materials described herein are intended to define the parameters of the various embodiments, they are by no means limiting and are merely exemplary. Many other embodiments will be apparent to those of skill in the art upon reviewing the above description. The scope of the various embodiments should, therefore, be determined with reference to the appended claims, along with the full scope of equivalents to which such claims are entitled. In the appended claims, the terms “including” and “in which” are used as the plain-English equivalents of the respective terms “comprising” and “wherein.” Moreover, in the following claims, the terms “first,” “second,” and “third,” etc. are used merely as labels, and are not intended to impose numerical requirements on their objects. Forms of term “based on” herein encompass relationships where an element is partially based on as well as relationships where an element is entirely based on. Forms of the term “defined” encompass relationships where an element is partially defined as well as relationships where an element is entirely defined. Further, the limitations of the following claims are not written in means-plus-function format and are not intended to be interpreted based on 35 U.S.C. § 112, sixth paragraph, unless and until such claim limitations expressly use the phrase “means for” followed by a statement of function void of further structure. It is to be understood that not necessarily all such objects or advantages described above may be achieved in accordance with any particular embodiment. Thus, for example, those skilled in the art will recognize that the systems and techniques described herein may be embodied or carried out in a manner that achieves or optimizes one advantage or group of advantages as taught herein without necessarily achieving other objects or advantages as may be taught or suggested herein.

While the invention has been described in detail in connection with only a limited number of embodiments, it should be readily understood that the invention is not limited to such disclosed embodiments. Rather, the invention can be modified to incorporate any number of variations, alterations, substitutions or equivalent arrangements not heretofore described, but which are commensurate with the spirit and scope of the invention. Additionally, while various embodiments of the invention have been described, it is to be understood that aspects of the disclosure may include only some of the described embodiments. Accordingly, the invention is not to be seen as limited by the foregoing description, but is only limited by the scope of the appended claims.

1. An ultrasound signal generating circuit comprising:
a control circuit having a control signal generating circuit;
a low power signal driver circuit for providing a low power pulse signal;
a high power signal driver circuit for providing a high power pulse signal; and
a radiofrequency switch configured to transmit the low power pulse signal and the high power pulse signal to an ultrasound probe for driving the ultrasound probe, wherein the radiofrequency switch isolates the low power signal driver circuit from the high power signal driver circuit;
wherein the ultrasound signal generating circuit is configured so that the radiofrequency switch transmits an output low power pulse signal output by the low power signal driver circuit or an output high power pulse output by the high power signal driver circuit based on an output of the control signal generating circuit.

2. The ultrasound signal generating circuit of claim 1, wherein the low power signal driver circuit outputs the low power pulse signal to a low power pulse signal transmission channel, and wherein the high power signal driver circuit outputs the high power pulse signal to a high power pulse signal transmission channel, wherein the radiofrequency switch outputs a propagating signal propagating on the low power pulse signal transmission channel or propagating signal propagating on the high power pulse signal transmission channel based on the output of the control signal generating circuit.

3. The ultrasound signal generating circuit of claim 1, wherein the ultrasound signal generating circuit includes an isolator circuit to isolate the radiofrequency switch from the control circuit.

4. The ultrasound signal generating circuit of claim 1, wherein the ultrasound signal generating circuit includes an isolator circuit to isolate the radiofrequency switch from the control circuit, wherein the isolator signal is configured to output a floating voltage control signal to the radiofrequency switch based on a control signal received from the control circuit.

5. The ultrasound signal generating circuit of claim 1, wherein the control circuit synchronizes switching between a first mode and a second mode by transmitting a deactivation signal to deactivate the a low power signal driver circuit, sensing a channel signal using a amplitude limiter for determining whether the low power signal driver circuit has been deactivated, and responsively to the determining transmitting an activation signal to activate the low power signal driver circuit, wherein the control circuit is restricted from transmitting the activation signal unless by the determining it is determined that the low power signal driver circuit has been deactivated.

6. The ultrasound signal generating circuit of claim 1, wherein an output of the radiofrequency switch defines an output of the ultrasound signal generating circuit.

7. The ultrasound signal generating circuit of claim 1, configured for receipt of an ultrasound probe of a candidate set of ultrasound probes, wherein the control signal generating circuit outputs a first control signal based on a first ultrasound transducer probe being attached to the ultrasound signal generating circuit, and wherein the control signal generating circuit outputs a second control signal based on second ultrasound probe being attached to the ultrasound signal generating circuit.

8. The ultrasound signal generating circuit of claim 1, wherein a control signal output by the control signal generating circuit is based on user defined data input by a user using a manually operated user interface.

9. The ultrasound signal generating circuit of claim 1, wherein a control signal output by the control signal generating circuit is based on an ultrasound transducer probe attached to the ultrasound signal generating circuit, and wherein the control signal is based on user defined data input by a user using a manually operated user interface.

10. The ultrasound signal generating circuit of claim 1, wherein the radiofrequency switch is a single pole double throw switch.

11. The ultrasound signal generating circuit of claim 1, wherein the radiofrequency switch includes a first power transistor pair and a second power transistor pair, the first power transistor pair connected in a back to back configuration characterized by sources of transistors defining the first power transistor pair being connected together, the second power transistor pair connected in a back to back configuration characterized by sources of transistors defining the second power transistor pair being connected together, wherein the first power transistor pair transmits the low power ultrasound signal based on the first power transistor pair being in an ON state, and wherein the second transistor pair transmits the high power ultrasound signal based on the second transistor pair being in an ON state.

12. The ultrasound signal generating circuit of claim 11, wherein the first power transistor pair and the second power transistor pair are provided by MOSFETs.

13. The ultrasound signal generating circuit of claim 11, wherein a transistor of the first power transistor pair is a transistor selected from the group consisting of silicon based MOSFET and a GaN based MOSFET.

14. The ultrasound signal generating circuit of claim 1, wherein the radiofrequency switch includes a first power transistor pair and a second power transistor pair, wherein each of the first power transistor pair and the second power transistor pair are configured to provide bi-directional power flow.

15. The ultrasound signal generating circuit of claim 1, wherein the radiofrequency switch includes a first power transistor pair and a second power transistor pair, the first power transistor pair having sources connected together and gates connected together, the second power transistor pair having sources connected together and gates connected together, wherein the first power transistor pair transmits the low power ultrasound signal based on the first power transistor pair being in an ON state, and wherein the second transistor pair transmits the high power ultrasound signal based on the second transistor pair being in an ON state.

16. The ultrasound signal generating circuit of claim 1, wherein the radiofrequency switch includes a power transistor pair, wherein sources of transistors defining the transistor pair are connected at a common node configured as a floating voltage node.

17. The ultrasound signal generating circuit of claim 1, wherein the ultrasound signal generating circuit includes an isolator circuit to isolate the radiofrequency switch from the control circuit, wherein the isolator circuit includes a low voltage isolator transformer.

18. The ultrasound signal generating circuit of claim 1, wherein the radiofrequency switch transmits a return signal attributable to a reflected ultrasound signal.

19. The ultrasound signal generating circuit of claim 1, wherein the radiofrequency switch transmits a return signal attributable to a reflected ultrasound signal to a signal processing circuit based on the first power transistor pair being in an ON state.

20. The ultrasound signal generating circuit of claim 1, wherein the radiofrequency switch includes a first power transistor pair and a second power transistor pair, and wherein respective analog gate drivers are used to amplify a control signal provided by respective isolator circuits to drive the first power transistor pair and the second power transistor pair.

21. The ultrasound signal generating circuit of claim 1, wherein the radiofrequency switch includes a power transistor pair, and wherein sources of transistors defining the transistor pair are connected at a common node configured as a floating voltage node, wherein the ultrasound signal generating circuit includes an isolator circuit to isolate the radiofrequency switch from the control circuit, wherein the isolator signal is configured to output a floating voltage control signal to common node based on a control signal received from the control circuit.

22. The ultrasound signal generating circuit of claim 1, wherein the ultrasound signal generating circuit includes an isolator circuit to isolate the radiofrequency switch from the control circuit, wherein the radiofrequency switch includes a first power transistor pair and a second power transistor pair, the first power transistor pair connected in a back to back configuration characterized by sources of transistors defining the first power transistor pair being connected together, the second power transistor pair connected in a back to back configuration characterized by sources of transistors defining the second power transistor pair being connected together, wherein the first power transistor pair transmits the low power ultrasound signal based on the first power transistor pair being in an ON state, and wherein the second transistor pair transmits the high power ultrasound signal based on the second transistor pair being in an ON state.

23. The ultrasound signal generating circuit of claim 1, wherein the ultrasound signal generating circuit includes an isolator circuit to isolate the radiofrequency switch from the control circuit, wherein the radiofrequency switch includes a first power transistor pair and a second power transistor pair, the first power transistor pair having sources connected together and gates connected together, the second power transistor pair having sources connected together and gates connected together, wherein the first power transistor pair transmits the low power ultrasound signal based on the first power transistor pair being in an ON state, and wherein the second transistor pair transmits the high power ultrasound signal based on the second transistor pair being in an ON state.
